(19) United States
(12) Patent Application Publication
(10) Pub. No.: US 2020/0000642 A1
(43) Pub. Date: Jan. 2, 2020

(54) MULTILAYER ABSORBENT DRESSING CONSTRUCTION

(71) Applicant: Systagenix Wound Management, Limited, West Sussex (GB)

(72) Inventor: Alexander Waite, Keighley (GB)

(73) Assignee: Systagenix Wound Management, Limited, West Sussex (GB)

(21) Appl. No.: 16/446,151

(22) Filed: Jun. 19, 2019

Related U.S. Application Data
(60) Provisional application No. 62/691,344, filed on Jun. 28, 2018.

(51) Int. Cl. A61F 13/02

(52) U.S. Cl. CPC A61F 13/022; A61F 13/0209; A61F 13/0289; A61F 13/025; A61F 13/0213; A61F 13/0243

(57) ABSTRACT

A wound dressing includes a first elastic foam layer, a superabsorbent layer, and a second elastic foam layer. The first elastic foam layer is configured to engage a wound bed and has a first side and a second side, the second side configured to face the wound surface. The superabsorbent layer is configured to absorb wound fluid and has a first side and a second side, the second side configured to face the first side of the elastic foam layer. The second elastic foam layer has a first side and a second side, the second side configured to face the first side of the superabsorbent layer.

### CROSS-REFERENCE TO RELATED APPLICATIONS

This application claims the benefit of priority to U.S. Provisional Application No. 62/691,344, filed on Jun. 28, 2018, which is incorporated herein by reference in its entirety.

### BACKGROUND

The present disclosure relates generally to a wound dressing. The present disclosure relates more particularly to a wound dressing having one or more elastic foam layers and a superabsorbent layer, wherein the superabsorbent layer combines with the elastic foam layer(s) to increase the absorption of wound fluid by the wound dressing.

It is often desirable to remove fluid from a wound bed to promote healing of the wound. In some cases, wound dressings include a layer of superabsorbent material configured to increase absorptivity of fluid from the wound bed. However, super absorbent layers within the wound dressing often times reduce the overall elasticity or flexibility of the wound dressing, which may negatively affect the conformability, adherence, and comfort during wear time. An absorbent wound dressing that retains the elasticity of a foam dressing, but that includes superabsorbent material for enhanced absorption of wound exudate, may be beneficial.

### SUMMARY

One implementation of the present disclosure is a wound dressing including a first elastic foam layer, a superabsorbent layer, and a second elastic foam layer. The first elastic foam layer is configured to engage a wound bed, and has a first side and a second side, the second side configured to face the wound surface. The superabsorbent layer is configured to absorb wound fluid and has a first side and a second side, the second side configured to face the first side of the first elastic foam layer. The second elastic foam layer has a first side and a second side, the second side configured to face the first side of the superabsorbent layer.

Another implementation of the present disclosure is a method of making a wound dressing. The method includes providing a first foam layer having a first side and a second side, the second side configured to face the wound surface. The method further includes providing a superabsorbent layer on at least a portion of the first side of the first foam layer. The method further includes providing a second foam layer over the superabsorbent layer. Finally, the method includes bonding the edges of the first foam layer and the second foam layer with the superabsorbent layer unbounded and free-floating between the first foam layer and the second foam layer.

Another implementation of the present disclosure is a wound dressing including an elastic foam layer, a plurality of superabsorbent pads, and a backing layer. The elastic foam layer is configured to engage a wound bed and has a first side and a second side, the second side configured to face the wound surface, wherein the elastic foam layer has a plurality of openings on the first side. The plurality of superabsorbent pads are configured to absorb wound fluid and fit within the openings of the elastic foam layer. The backing layer has a first side and a second side, the second side configured to face the first side of the elastic foam layer.

Another implementation of the present disclosure is a method of making a wound dressing. The method includes providing a foam layer having a first side and a second side, the first side having a pattern of pockets provided thereon, and the second side configured to face the wound surface. The method further includes depositing a superabsorbent material into the pockets on the first side of the first foam layer to create superabsorbent pockets. Finally, the method includes applying a backing film over the first side of the foam layer and the superabsorbent pockets.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a top view of a wound dressing, according to an exemplary embodiment.

FIG. 2 is a bottom view of the wound dressing of FIG. 1, according to an exemplary embodiment.

FIG. 3 is an exploded view illustrating several layers of the wound dressing of FIGS. 1 and 2, according to an exemplary embodiment.

FIG. 4 is an exploded view illustrating several layers of the wound dressing of FIGS. 1 and 2, according to an exemplary embodiment.

FIG. 5 is an exploded view illustrating several layers of a wound dressing, according to an exemplary embodiment.

FIG. 6 is a cross-sectional view of a wound dressing adhered to a surface, according to an exemplary embodiment.

FIG. 7 is a cross-sectional view of a wound dressing adhered to a surface, according to an exemplary embodiment.

### DETAILED DESCRIPTION

### Overview

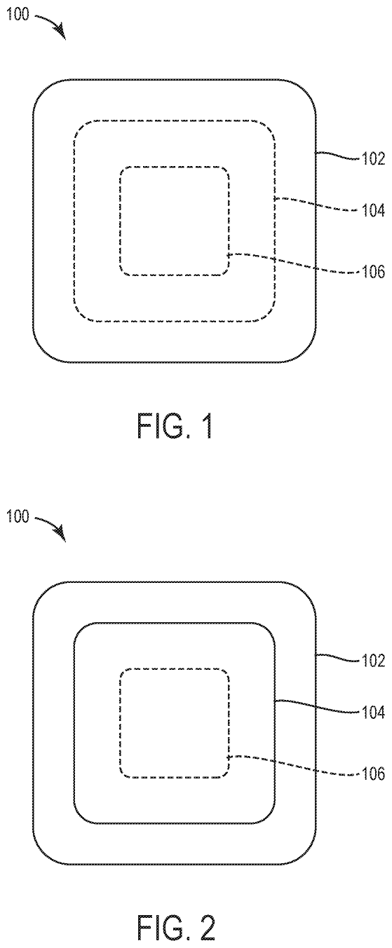

Referring generally to the FIGS. 1-7, a wound dressing with an elastic foam layer and a superabsorbent layer is shown, according to exemplary embodiments. The wound dressing of the exemplary embodiments has multiple layers including one or more elastic foam layers, a superabsorbent layer, and a backing layer configured to overlay a periwound surrounding the wound bed. In some embodiments, the superabsorbent layer is in the form of a plurality of non-contiguous superabsorbent pads.

Advantageously, the superabsorbent layer increases the absorptivity of the wound dressing while retaining elasticity and flexibility of the wound dressing. In some embodiments, the superabsorbent layer is placed between a first elastic foam layer and a second elastic foam layer. The first elastic foam layer and the second elastic foam layer may be bonded together around the superabsorbent layer, while the superabsorbent layer is “freefloating” within and not bound to either of the first elastic foam layer or the second elastic foam layer, such that the elastic foam layers can flex and stretch around the superabsorbent layer, retaining elasticity of the wound dressing.

In some embodiments, the superabsorbent layer comprises a single sheet of a superabsorbent material. In other embodiments, the superabsorbent layer has a plurality of fenestrations or slits extending from a first side of the superabsorbent layer to a second side of the superabsorbent layer. Fenestrations or slits within the superabsorbent layer introduce a degree of “give” or flexibility in the superabsorbent layer to reduce an overall rigidity of the wound dressing. In other embodiments, the superabsorbent layer may be provided in the form of a powder or particulate material that is contained within an area between the first elastic foam layer and the second elastic foam layer. A super absorbent layer in such a granular form has enhanced flexibility, thus reducing overall rigidity of the wound dressing.

In still other embodiments, the superabsorbent layer may comprise a plurality of superabsorbent pads placed into a plurality of openings on the first side of the second elastic foam layer. The pads may be arranged in a non-contiguous manner such as in the form of “islands” or the like, and separated from one another by an intervening or surrounding region of a foam layer. Such an embodiment may not require the first elastic foam layer. In some embodiments, a backing layer may be placed over the superabsorbent pads and the first elastic foam layer. In some embodiments, the superabsorbent pads are configured to swell upon absorbing the wound exudate fluid, which provides a visual indication of which portion of the wound dressing has absorbed wound exudate and/or when the wound dressing has absorbed a maximum capacity of wound exudate. This is intended to prevent unnecessary changing of the wound dressing and provides an indication to a caregiver as to when a wound dressing should be replaced. Additional features and advantages of the wound dressing are described in detail below.

### Wound Dressing

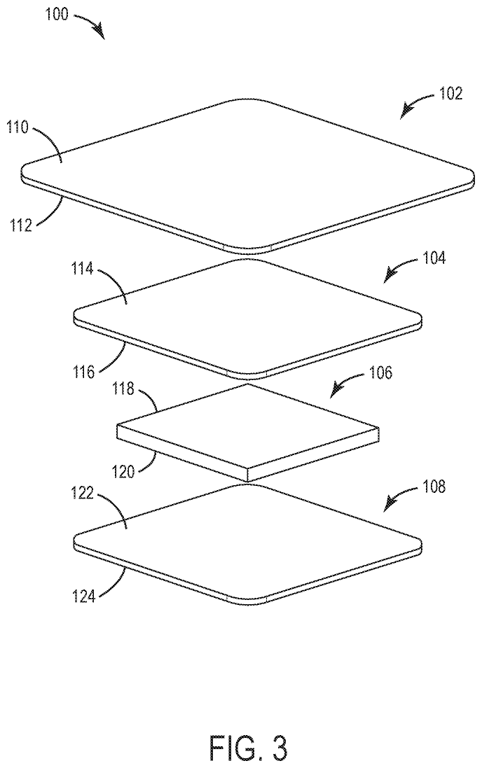

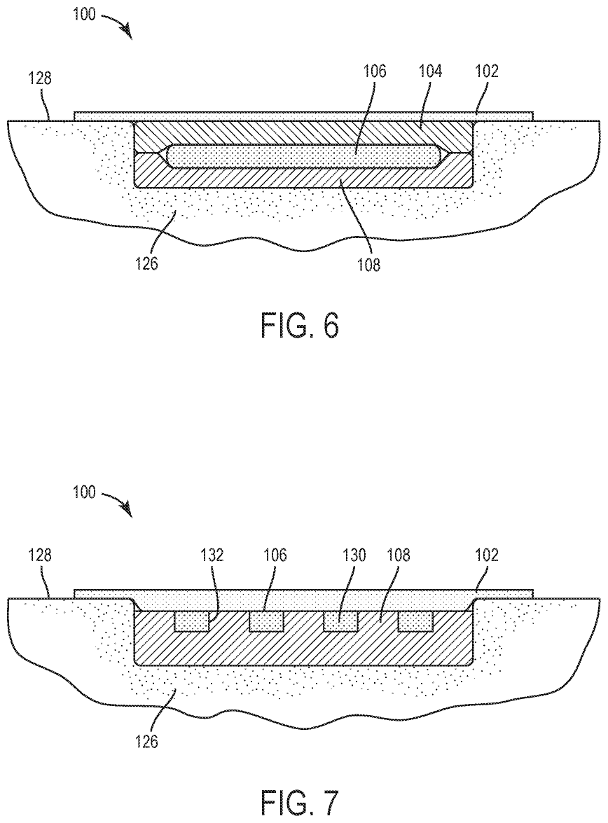

Referring now to FIGS. 1-7, a wound dressing 100 is shown, according to exemplary embodiments. In brief overview, FIG. 1 is a top view of wound dressing 100 as would be visible when wound dressing 100 is adhered to a surface (e.g., a patient's skin). FIG. 2 is a bottom view of wound dressing 100 showing the wound-contacting surface of wound dressing 100. The broken lines in FIG. 1 outline the layers of wound dressing 100 that are not visible in that view. FIGS. 3-5 are exploded views illustrating several layers 102-108 of wound dressing 100 according to different embodiments. FIGS. 6-7 schematically illustrate different embodiments of a cross-sectional view of wound dressing 100 adhered to a patient at a wound bed 126 and periwound 132.

In various embodiments, wound dressing 100 can be formed as a substantially flat sheet for topical application to wounds, or formed as a contoured dressing for application to body surfaces having high curvature. The size of wound dressing 100 can vary depending on the size of the wound to be dressed. For example, it is contemplated that the size of wound dressing 100 can range from approximately 1 cm² to 200 cm², and more preferably from approximately 4 cm² to 100 cm². However, other shapes and sizes of wound dressing 100 are also possible depending on the intended use. In other embodiments, wound dressing 100 may have a substantially convex or concave shape, or other customizable topography to adhere to wounds located on areas such as the knee or elbow.

Wound dressing 100 is shown to include (among other possible layers) a backing layer 102, an optional first elastic foam layer 104, a superabsorbent layer 106, and a second elastic foam layer 108. In some embodiments, wound dressing 100 has only backing layer 102, superabsorbent layer 106, and second elastic foam layer 108. In the embodiments shown in FIGS. 6-7, wound dressing 100 is applied for use in connection with a wound bed 126, with the backing layer 102 adhering to the patient surrounding a periwound 128. In other embodiments, the layers 102-108 may lie entirely within the confines of the periwound 128. However, other configurations of the locations of the layers are also possible depending on the intended use, including the removal of backing layer 102 from the wound dressing 100.

### Backing Layer

In some embodiments, wound dressing 100 includes a backing layer 102. Backing layer 102 is shown to include a first side 110 and a second, wound-facing side 112 opposite first side 110. When wound dressing 100 is applied to a wound, first side 110 faces away from the wound whereas second side 112 faces toward the wound. Drape layer 102 attaches over first elastic foam layer 104, superabsorbent layer 106, and second elastic foam layer 108 and provides a barrier to passage of microorganisms through wound dressing 100. In some embodiments, backing layer 102 is a thin layer of polyurethane film. One example of a suitable material for backing layer 102 is the polyurethane film known as ESTANE 5714F. Other suitable polymers for forming backing layer 102 include poly alkoxyalkyl acrylates and methacrylates, such as those described in Great Britain Patent Application No. 1280631A filed Nov. 22, 2002, the entire disclosure of which is incorporated by reference herein. In some embodiments, backing layer 102 includes a continuous layer of a high-density blocked polyurethane foam that is predominantly closed-cell. Backing layer 102 may have a thickness in the range of 10 μm to 100 μm, preferably in the range of 50 μm to 70 μm.

Backing layer 102 may be substantially impermeable to liquid and substantially permeable to moisture vapor. In other words, backing layer 102 may be permeable to water vapor, but not permeable to liquid water or wound exudate. This increases the total fluid handling capacity (TFHC) of wound dressing 100 while promoting a moist wound environment. In some embodiments, backing layer 102 is also impermeable to bacteria and other microorganisms. In some embodiments, backing layer 102 is configured to wick moisture from first elastic foam layer 104 and distribute the moisture across first side 110. Backing layer 102 may have a high moisture vapor transmission rate (MVTR) to wick moisture from the wound dressing 100 so that a moisture gradient is established from the wound bed 126, outwardly through the dressing and to the surrounding atmosphere. In some embodiments, the material of backing layer 102 may alter the breathability and MVTR of backing layer 102 to meet the specific requirements of the particular wound dressing 100.

Second side 112 of backing layer 102 may be partially or entirely coated with an acrylic or other adhesive. The adhesive applied to second side 112 ensures that wound dressing 100 adheres to the skin around the periwound 128 and that wound dressing 100 remains in place throughout the wear time. In the embodiment shown, the perimeter of backing layer 102 extends beyond (e.g., circumscribes) the perimeters of first elastic foam layer 104, superabsorbent layer 106, and second elastic foam layer 108 to provide an adhesive-coated margin for adhering wound dressing 100 to the skin of a patient adjacent to the wound being treated, shown in FIGS. 6-7 as periwound 128. The adhesive-coated margin may extend around all sides of first elastic foam layer 104, superabsorbent layer 106, and second elastic foam layer 108 such that wound dressing 100 is a so-called “island” dressing. In other embodiments, the adhesive-coated margin can be eliminated and wound dressing 100 can be applied using other techniques.

In some embodiments, second side 112 of backing layer 102 contacts first side 114 of first elastic foam layer 104. Second side 112 of backing layer 102 may be adhered to first side 114 of first elastic foam layer 104 or may simply contact first side 114 without the use of an adhesive. In some embodiments, the adhesive applied to second side 112 of backing layer 102 is moisture vapor transmitting and/or patterned to allow passage of water vapor therethrough. The adhesive may include a continuous moisture vapor transmission, pressure-sensitive adhesive layer of the type conventionally used for island-type wound dressings (e.g., a polyurethane-based pressure sensitive adhesive). One example of an adhesive which can be used is a pressure sensitive adhesive based on acrylate ester copolymers, polyvinyl ethyl ether and polyurethane, as described in Great Britain Patent Application No. 1280631A. The basis weight of the adhesive may be 20 to 250 g/m², and more preferably 50 to 150 g/m². In other embodiments, the adhesive may be an acrylic, silicone, polyurethane hydrogel, or any combination thereof. The adhesive may be distributed as a substantially continuous coat on second side 112 of backing layer 102, or may be coated in a pattern onto second side 112 of backing layer 102.

### First Elastic Foam Layer

In some embodiments, wound dressing 100 includes a first elastic foam layer 104. First elastic foam layer 104 is shown to include a first side 114 and a second, wound-facing side 116 opposite first side 114. When wound dressing 100 is applied to a wound, first side 114 faces away from the wound whereas second side 116 faces toward the wound. In some embodiments, first side 114 of first elastic foam layer 104 contacts second side 112 of backing layer 102. Similarly, second side 116 of first elastic foam layer 104 may contact first side 118 of superabsorbent layer 106. In some embodiments, a perimeter of second side 116 may contact first side 122 of second elastic foam layer 108. In some embodiments, first elastic foam layer 104 is not bound, adhered, laminate, or otherwise attached to superabsorbent layer 106. In some embodiments, a perimeter of second side 116 of first elastic foam layer 104 is laminated or otherwise bound to a perimeter of a first side 122 of second elastic foam layer 108, such that superabsorbent layer 106 is encapsulated between first elastic foam layer 104 and second elastic foam layer 108 in a free-floating manner. First elastic foam layer 104 and second elastic foam layer 108 can stretch as necessary around superabsorbent layer 106 and superabsorbent layer 106 can move between first elastic foam layer 104 and second elastic foam layer 108.

In some embodiments, first elastic foam layer 104 may comprise a polyurethane foam or a polyethylene foam. In some embodiments, first elastic foam layer 104 includes a flexible plasticized hydrophilic polymer matrix having an internal cellular structure. Several examples of hydrophilic foams which can be used to make first elastic foam layer 104 are described in detail un U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein. In other embodiments, first elastic foam layer 104 may be formed from aromatic or aliphatic precursors. Advantageously, first elastic foam layer 104 may provide enhanced absorbency for liquid exudate. This is because the porous structure of the foam provides for rapid uptake of liquid exudate.

In some embodiments, first elastic foam layer 104 includes an antimicrobial agent, anti-inflammatory agent, or other active agents to promote effective wound healing. Non-limiting examples of such active materials may include antimicrobial silver, silver oxidized regenerated cellulose (ORC) (e.g., approximately 25 wt % ionically bound silver), polyhexamethylene biguanide (PHMB), nonsteroidal ant-inflammatory drugs such as acetaminophen, steroids, anti-inflammatory cytokines, anesthetics, antimicrobial agents such as penicillin or streptomycin, antiseptics such as chlorhexidine, growth factors such as fibroblast growth factor (FGF), a platelet derived growth factor (PDGF), or an epidermal growth factor (EGF), and other therapeutic agents, individually or in any combination. If present, such active materials may typically be included at any effective level that show therapeutic efficacy, while preferably not being at such a high level as to significantly counteract any critical or desired physical, chemical, or biological property of the dressing. Depending upon the therapeutic goal, any active material may be loaded at a level of from about 10 wppm to about 10 wt % of the layer in which it is present, for example, from about 50 wppm to about 5 wt % or from about 100 wppm to about 1 wt %. The active material may be contained within a thin film on second side 116 of first elastic foam layer 104 or may be distributed within first elastic foam layer 104.

### Superabsorbent Layer

In some embodiments, wound dressing 100 includes a superabsorbent layer 106. Superabsorbent layer 106 is shown to include a first side 118 and a second, wound-facing side 120 opposite first side 118. When wound dressing 100 is applied to a wound, first side 118 faces away from the wound whereas second side 120 faces toward the wound. In some embodiments, first side 118 of superabsorbent layer 106 contacts second side 116 of first elastic foam layer 104. In other embodiments, wound dressing 100 is free of first elastic foam layer 104, and first side 118 of superabsorbent layer 106 contacts second side 112 of backing layer 102. Similarly, second side 120 of superabsorbent layer 106 may contact first side 122 of second elastic foam layer 108. In some embodiments, superabsorbent layer 106 is configured to wick moisture from second elastic foam layer 108 and distribute the moisture across first side 118. In some embodiments, superabsorbent layer 106 is encapsulated between first elastic foam layer 104 and second elastic foam layer 108 in a “loose” or free-floating manner. Superabsorbent layer 106 is smaller in dimension than either of the first elastic foam layer 104 and second elastic foam layer 108 such that the perimeter of first elastic foam layer 104 and second elastic foam layer 108 both extend beyond the perimeter of superabsorbent layer 106. First side 118 of superabsorbent layer 106 is not adhered to second side 116 of first elastic foam layer 104. Similarly, second side 120 of superabsorbent layer 106 is not adhered to first side 122 of second elastic foam layer 108. Superabsorbent layer 106 is substantially free-floating between first elastic foam layer 104 and second elastic foam layer 108 such that first elastic foam layer 104 and second elastic foam layer 108 are independently stretchable relative to and around superabsorbent layer 106.

In some embodiments, superabsorbent layer 106 includes a hydrogel or hydrogel composition. Several examples of hydrogels and hydrogel compositions which can be used to form superabsorbent layer 106 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein.

The expressions “hydrogel” and “hydrogel compositions” used herein are not to be considered as limited to gels which contain water, but extend generally to all hydrophilic gels and gel compositions, including those containing organic non-polymeric components in the absence of water. For example, superabsorbent layer 106 may be formed from a polyurethane that entraps water to form a gel. In some embodiments, superabsorbent layer 106 is substantially continuous and/or substantially non-porous or non-foamed. Superabsorbent layer may include a flexible plasticized hydrophilic polymer matrix having a substantially continuous internal structure. The density of superabsorbent layer 106 may be greater than 0.5 g/cm³, more preferably greater than 0.8 g/cm³, and most preferably from 0.9 to 1.1 g/cm³. In some embodiments, the thickness of superabsorbent layer 106 is from 1 mm to 10 mm, more preferably from 2 mm to 5 mm. In still other embodiments, superabsorbent layer 106 includes a plurality of one or more cellulosic gelling fibers including carboxymethyl cellulose, carboxyethl cellulose, hydroxyethyl cellulose, hydroxypropyl cellulose, hydroxypropyllmethyl cellulose, or cellulose ethyl sulphonate. In still other embodiments, superabsorbent layer 106 includes a nonwoven hydrofiber material. In other embodiments, superabsorbent layer includes one or more of a plurality of reinforcing fibers comprising a polyurethane gel, an amide polymer, an olefin polymer, an ester polymer, or a modified acrylamide polymer. The material, formulation, and size of superabsorbent layer 106 may be determined based on desired fluid handling requirements of wound dressing 100.

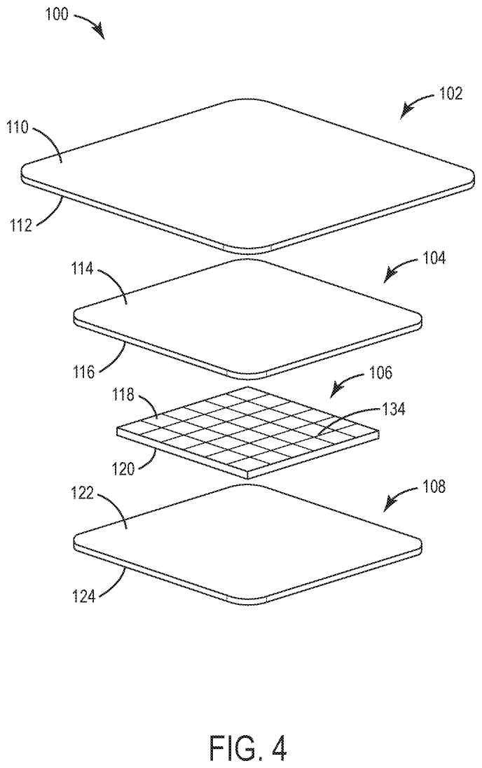

In some embodiments, superabsorbent layer 106 is a continuous sheet or layer of material such as shown in FIG. 3. In other embodiments, superabsorbent layer 106 includes a plurality of fenestrations 134 such as shown in FIG. 4. Fenestrations 134 may extend from first side 118 to second side 120 of superabsorbent layer 106. In other embodiments, fenestrations 134 may extend only a portion of the way through superabsorbent layer 106. In some embodiments, fenestrations 134 may extend linearly across superabsorbent layer 106. In other embodiments, fenestrations 134 may extend in a multidirectional pattern across superabsorbent layer 106. In some embodiments, fenestrations 134 may be substantially evenly distributed in a grid pattern upon a portion of superabsorbent layer 106. In other embodiments, fenestrations 134 may be distributed in an uneven (e.g. non-uniform, random, etc.) pattern across superabsorbent layer 106. Further, fenestrations 134 may be arranged in a plurality of patterns. The inclusion of fenestrations 134 within superabsorbent layer 106 may introduce a degree of “give” or flexibility in the material of superabsorbent layer 106 in an amount corresponding to the depth, spacing, and pattern of the fenestrations 134, acting to improve overall flexibility of wound dressing 100. In some embodiments, superabsorbent layer 106 with fenestrations 134 is still free-floating and not laminated or adhered to either of first elastic foam layer 104 or second elastic foam layer 108.

In some embodiments, superabsorbent layer 106 may be in the form of a superabsorbent particulate or powder material. The particulate may be placed centrally between first elastic foam layer 104 and second elastic foam layer 108, such that the particulate is encapsulated within first elastic foam layer 104 and second elastic foam layer 108. Superabsorbent layer 106 may have a granular composition, such that the particulate is not held together by entanglement, weaving/knot, or lamination. Superabsorbent layer 106 in the form of a particulate or powder increases the flexibility of superabsorbent layer 106, which may act to reduce the overall rigidity of wound dressing 100.

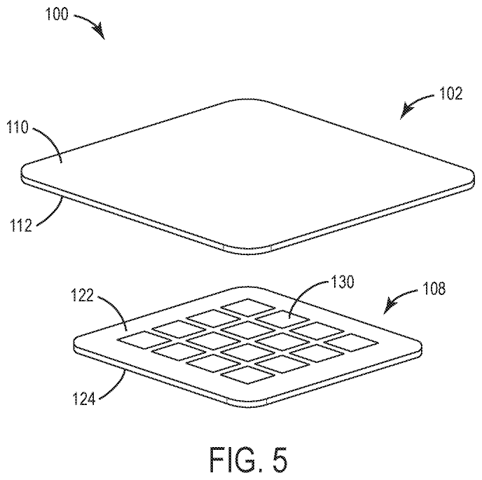

In the embodiment shown in FIGS. 5 and 7, superabsorbent layer 106 may be in the form of a plurality of non-contiguous superabsorbent “pads” 130. Superabsorbent pads 130 may be placed into openings 132 within first side 122 of second elastic foam layer 108. In some embodiments, superabsorbent pads 130 are adhered to second elastic foam layer 108 within openings 132. Superabsorbent pads 130 may be sized appropriately as to fit within openings 132 such that a top portion of superabsorbent pads 130 lie flush with first side 122 of second elastic foam layer 108. In other embodiments, superabsorbent pads 130 may be larger than openings 132, such that a top of the superabsorbent pads 130 extends beyond first side 122 of second elastic foam layer 108. In some embodiments, superabsorbent pads 130 form a substantially even pattern within first side 122 of second elastic foam layer 108. In other embodiments, superabsorbent pads 130 are distributed in an uneven (e.g., non-uniform, random, etc.) pattern across first side 122. Superabsorbent pads 130 may be arranged in a non-contiguous manner such as in the form of “islands” or the like, and separated from one another by an intervening or surrounding region of a foam layer. Superabsorbent pads 130 may be adhered or otherwise laminated to second side 112 of backing layer 102. In other embodiments, superabsorbent pads 130 may contact second side 112 of backing layer 102 without the use of adhesive.

Superabsorbent pads 130 are configured to swell upon absorption of fluid from wound bed 126. Superabsorbent pads 130 may be more hydrophilic than second elastic foam layer 108, such that superabsorbent pads 130 assist in wicking fluid from wound bed 126 and second side 124 of second elastic foam layer 108 towards first side 122 and into superabsorbent pads 130. Superabsorbent pads 130 may provide a visual indication as to when wound dressing 100 has reached a maximum fluid absorption capacity and needs to be changed. The visual indication of superabsorbent pads 130 swelling may be apparent without the removal of wound dressing 100, such that backing layer 102 also has a change in appearance or surface texture.

### Second Elastic Foam Layer

In some embodiments, wound dressing 100 includes a second elastic foam layer 108. Second elastic foam layer 108 is shown to include a first side 122 and a second, wound-facing side 124 opposite first side 122. When wound dressing 100 is applied to a wound, first side 122 faces away from the wound whereas second side 124 faces toward and may contact the wound, such as wound bed 126. In some embodiments, first side 122 of second elastic foam layer 108 contacts second side 120 of superabsorbent layer 106. In some embodiments, a perimeter of first side 122 may also contact second side 122 of first elastic foam layer 104. Similarly second side 124 of second elastic foam layer 108 contacts wound bed 126. In some embodiments, second elastic foam layer 108 is not bound, adhered, laminated, or otherwise attached to superabsorbent layer 106. In some embodiments, a perimeter of first side 122 is laminated or otherwise bound to a perimeter of second side 116 of first elastic foam layer 104, such that superabsorbent layer 106 is encapsulated between first elastic foam layer 104 and second elastic foam layer 108, allowing wound dressing 100 to stretch around superabsorbent layer 106.

In some embodiments, second elastic foam layer 108 may comprise a polyurethane foam or a polyethylene foam, such as described above with reference to first elastic foam layer 104. In some embodiments, second elastic foam layer 108 includes an antimicrobial agent, anti-inflammatory agent, or other active agent distributed across second side 124, as described about with reference to first elastic foam layer 104. In some embodiments, second elastic foam layer 108 may comprise substantially the same material, composition, and size as first elastic foam layer 104. In still other embodiments, second elastic foam layer 108 may comprise a differing material, composition, or size as first elastic foam layer 104.

In some embodiments, first side 122 of second elastic foam layer 108 includes a plurality of openings 132, as shown in FIG. 7. Openings 132 may extend from first side 122 into a central portion of second elastic foam layer 108. In other embodiments, openings 132 may extend from first side 122 to second side 124, forming a plurality of holes through second elastic foam layer 108. Openings 132 may be substantially evenly distributed upon at least a portion of first side 122 of second elastic foam layer 108. In other embodiments, openings 132 may be distributed in an uneven (e.g., non-uniform, random, etc.) pattern on first side 122. In some embodiments, openings 132 may vary in depth, width, and distribution within first side 122. Openings 132 are configured to receive superabsorbent pads 130. In the embodiment of FIG. 7, wound dressing 100 includes only backing layer 102, superabsorbent layer 106 in the form of superabsorbent pads 130, and second elastic foam layer 108. Superabsorbent pads 130 may be sized to fit within openings 132 such that the tops of superabsorbent pads 130 lie flush with first side 122 of second elastic foam layer 108. In one embodiment, both the tops of superabsorbent pads 130 and first side 122 may be adhered to second side 112 of backing layer 102. In other embodiments, only first side 122 may be adhered to second side 112 of backing layer 102, such that the superabsorbent pads 130 are encapsulated between backing layer 102 and second elastic foam layer 108. The encapsulation of superabsorbent pads 130 may allow for greater flexibility of wound dressing 100, as backing layer 102 and second elastic foam layer 108 are capable of stretching around superabsorbent pads 130.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

What is claimed is:

1. A wound dressing comprising:
a first elastic foam layer configured to engage a wound bed, and having a first side and a second side, the second side configured to face the wound surface;
a superabsorbent layer configured to absorb wound fluid and having a first side and a second side, the second side configured to face the first side of the first elastic foam layer; and
a second elastic foam layer having a first side and a second side, the second side configured to face the first side of the superabsorbent layer.

2. The wound dressing of claim 1, further comprising a backing layer adhered to the second elastic foam layer opposite the superabsorbent layer.

3. The wound dressing of claim 2, wherein the backing layer is substantially impermeable to liquid and substantially permeable to vapor.

4. The wound dressing of claim 2, wherein the backing layer extends beyond a perimeter of the second elastic foam layer to provide and adhesive-coated margin configured to adhere the wound dressing to a surface.

5. The wound dressing of claim 1, wherein the second elastic foam layer extends beyond a perimeter of the superabsorbent layer and the superabsorbent layer lies within a perimeter of the first elastic foam layer.

6. The wound dressing of claim 5, wherein the perimeter of the first elastic foam layer is laminated to the perimeter of the second elastic foam layer, such that the superabsorbent layer is encapsulated within the first elastic foam layer and the second elastic foam layer.

7. The wound dressing of claim 5, wherein the superabsorbent layer is substantially free-floating between the first elastic foam layer and the second elastic foam layer, so that the first elastic foam layer and the second elastic foam layer are independently stretchable relative to the superabsorbent layer.

8. The wound dressing of claim 1, wherein the superabsorbent layer comprises a flexible plasticized hydrophilic polymer matrix having a substantially continuous internal structure.

9. The wound dressing of claim 1, wherein the superabsorbent layer is configured to wick moisture from the first elastic foam layer and distribute the moisture across the first side of the superabsorbent layer.

10. The wound dressing of claim 1, wherein the superabsorbent layer comprises a nonwoven hydrofiber material.

11. The wound dressing of claim 1, wherein the superabsorbent layer comprises a plurality of cellulosic gelling fibers comprising at least one of carboxymethyl cellulose, carboxyethyl cellulose, hydroxyethyl cellulose, hydroxypropyl cellulose, hydroxypropylmethyl cellulose, or cellulose ethyl sulphonate.

12. The wound dressing of claim 1, wherein the superabsorbent layer comprises a plurality of reinforcing fibers comprising at least one of a polyurethane gel, an amide polymer, an olefin polymer, an ester polymer, or a modified acrylamide polymer.

13. The wound dressing of claim 1, wherein the superabsorbent layer is substantially free-floating between the first elastic foam layer and the second elastic foam layer, and comprises a plurality of linear fenestrations configured to decrease an overall rigidity of the wound dressing.

14. The wound dressing of claim 13, wherein the fenestrations comprises slits that are evenly distributed in a grid pattern upon at least a portion of the superabsorbent layer.

15. The wound dressing of claim 13, wherein the fenestrations are distributed in a non-uniform pattern on the superabsorbent layer.

16. The wound dressing of claim 13, wherein the fenestrations are multidirectional.

17. The wound dressing of claim 1, wherein the superabsorbent layer comprises a particulate or powder material that is centrally disposed between and unbonded to the first elastic foam layer and the second elastic foam layer.

18. The wound dressing of claim 1, wherein the first elastic foam layer and/or the second elastic foam layer comprises at least one of a polyurethane foam or a polyethylene foam.

19. The wound dressing of claim 1, wherein the first elastic foam layer and/or the second elastic foam layer comprises an antimicrobial agent.

20. The wound dressing of claim 1, wherein the first elastic foam layer and/or the second elastic foam layer comprises an anti-inflammatory agent.

21. A method of making a wound dressing comprising:
providing a first foam layer having a first side and a second side, the second side configured to face the wound surface;
providing a superabsorbent layer on at least a portion of the first side of the first foam layer;
providing a second foam layer over the superabsorbent layer; and
bonding the edges of the first foam layer and the second foam layer with the superabsorbent layer unbounded and free-floating between the first foam layer and the second foam layer.

22. The method of claim 21, wherein the superabsorbent layer is provided in the form of a substantially rigid sheet of superabsorbent material.

23. The method of claim 21, wherein the superabsorbent layer provided in the form of a sheet of superabsorbent material having a plurality of fenestrations that permit at least limited flexing of the sheet about the fenestrations.

24. The method of claim 21, wherein the superabsorbent layer is provided in the form of a powder or a particulate.

25. A wound dressing comprising:
an elastic foam layer configured to engage a wound bed, and having a first side and a second side, the second side configured to face the wound surface, wherein the elastic foam layer has a plurality of openings on the first side;
a plurality of superabsorbent pads configured to absorb wound fluid and fit within the openings of the elastic foam layer; and
a backing layer having a first side and a second side, the second side configured to face the first side of the elastic foam layer.

26. The wound dressing of claim 25, wherein the openings of the elastic foam layer are evenly distributed upon at least a portion of the first side of the elastic foam layer.

27. The wound dressing of claim 25, wherein the openings extend from the first side of the elastic foam layer to the second side of the elastic foam layer, forming a hole therethrough.

28. The wound dressing of claim 25, wherein the openings extend from the first side of the elastic foam layer to a central portion of the elastic foam layer, such that the second side of the elastic foam layer does not contain the openings.

29. The wound dressing of claim 25, wherein the superabsorbent pads fit within the openings such that a top of the superabsorbent pads lies substantially flush with the first side of the elastic foam layer.

30. The wound dressing of claim 25, wherein the superabsorbent pads comprise a flexible plasticized hydrophilic polymer matrix having a substantially continuous internal structure.

31. A method of making a wound dressing comprising:
providing a foam layer having a first side and a second side, the first side having a pattern of pockets provided thereon, and the second side configured to face the wound surface;
depositing a superabsorbent material into the pockets on the first side of the first foam layer to create superabsorbent pockets;
applying a backing film over the first side of the foam layer and the superabsorbent pockets.

32. The method of claim 31, wherein the superabsorbent pockets are non-contiguous and the portions of the foam layer disposed between the superabsorbent pockets permit stretching and flexing of the dressing between the superabsorbent pockets.

33. The method of claim 31, wherein the superabsorbent pockets are configured to swell and provide a visual and a tactile indication through the backing layer of a degree of fluid absorption by the dressing.
